(19) United States
(12) Patent Application Publication
(10) Pub. No.: US 2023/0263651 A1
(43) Pub. Date: Aug. 24, 2023

(54) Urinary bags emptying device for use on power wheelchairs

(71) Applicant: Dzianis PLIUTAU, Hampton, VA (US)

(72) Inventor: Dzianis PLIUTAU, Hampton, VA (US)

(21) Appl. No.: 18/111,544

(22) Filed: Feb. 18, 2023

Related U.S. Application Data
(60) Provisional application No. 63/311,631, filed on Feb. 18, 2022.

(51) Int. Cl. A61F 5/44; A61G 5/10

(52) U.S. Cl. CPC A61F 5/4405; A61G 5/1002

(57) ABSTRACT

An improved system that allows a disabled person driving a power wheelchair to independently empty the contents of urine collection bags into toilets or urinals with the help of tilt and driving functions of the power wheelchair and an accelerometer. The system comprises a self priming pump, a leg bag and a large capacity urine collection bag. The input of the pump is interconnected with the draining tubes of the urinary leg bag and the large capacity bag. The large capacity bag is positioned substantially lower than the leg bag to let the contents of the leg bag continuously drain into the large capacity bag by gravity. The discharge line output terminated with a check valve is positioned near the front of the footplates. A microcontroller with connected sensors is used to monitor urine weight in the large capacity bag and to ensure the urinary bags are connected.

### CROSS-REFERENCE TO RELATED APPLICATIONS

This application claims the benefit of provisional Pat. Application Ser. No. 63/311/631, filed 2022 Feb 18 by the present inventor.

### TECHNICAL FIELD

The present disclosure relates to urine collection devices, and more specifically to urinary bags which are utilized by persons confined to power wheelchairs. More particularly, the invention relates to a system and method for allowing an occupant of power wheelchair to automatically empty a urinary bag into a toilet receptacle or urinal without the aid of an attendant or assistant.

### BACKGROUND

The following is a tabulation of some prior art that presently appears relevant:

U. S. Patents

| Patent Number | Kind Code | Issue Date | Patentee |
|---|---|---|---|
| 3568217 | B1 | 1971-03-09 | Anderson |
| 3931650 | B1 | 1976-01-13 | Miller |
| 5397315 | B1 | 1995-03-14 | Schmidt |
| 6012181 | B1 | 2000-01-11 | Johnson |
| 6526603 | B1 | 2003-03-04 | Murphy |
| 6945965 | B2 | 2005-09-20 | Whiting |
| 7491194 | B1 | 2009-02-17 | Oliwia |

U. S. Patent Application Publications

| Publication Nr. | Kind Code | Publ. Date | Applicant |
|---|---|---|---|
| 2004/0230181 | A1 | 2004-11-18 | Cawood |

Foreign Patent Documents

| Foreign Doc. Nr. | Cntry Code | Pub. Date | App or Patentee |
|---|---|---|---|
| 2496605 | GB | 2013-05-22 | Wills |

### Other Nonpatent Publications

Shalini A.M. D′Souza, SoYeon Ann, and Amanda Bernsohn, “Development of a Reverse Gravity Leg Bag Emptier”, Resna Annual Conference 2009, https://www.resna.org/sites/default/files/legacy/conference/proceedings/2009/SDC2009/DSou za.html
YouTube Video, “Electric LegbBag Emptier Pinch Model”, Aug. 22, 2009, https://www.youtube.com/watch?v=fUpcpT2bxDw
YouTube Video, “Demonstration and Use of a Leg Bag Emptier”, Jan. 6, 2010, https://www.youtube.com/watch?v=rjONINu8IFQ
YouTube Video, “The Premier Leg Bag Emptier - The PUMP″, part 1, Aug. 26, 2011, https://www.youtube.com/watch?v=uwHF1uPaRsQ
YouTube Video, “The PUMP leg bag emptier - Installation Video”, part 2, Nov. 7, 2011, https://www.youtube.com/watch?v=2dcq2C3tNJU
YouTube Video, “Why Kan-Go-Wee?”, part 1, Oct. 31, 2013, https://www.youtube.com/watch?v=tZv5CEzDGKE
YouTube Video, “Kan Go Wee Installation (½)”, part 2, Oct. 31, 2013, https://www.youtube.com/watch?v=YPO9xmYHMG8
YouTube Video, “Kan Go Wee Attachment (2/2)”, part 3, Oct. 31, 2013, https://www.youtube.com/watch?v=Tf60AGFU-78
YouTube Video, “Falcon Rehab Electronic Leg Bag Drain”, Nov. 13, 2013, https://www.youtube.com/watch?v=VnKFuRJQ9T8
YouTube Video (channel has 6 more videos), “Melio Self-Emptying Leg Bag System”, Jul. 31, 2014, https://www.youtube.com/watch?v=bWDaSyGz88o
YouTube Video, “GoPee Installation”, Oct. 13, 2015, https://www.youtube.com/watch?v=hSl1FQGLlrQ
YouTube Video, “The all new Air Stream, leg bag emptier”, Oct 6. 2022, https://www.youtube.com/watch?v=iYVzduvXqkU
“JB-3 Leg Bag Emptying System”, http://www.jb-3.com/
“Freedom Flow wireless leg bag emptier”, https://www.freedom-flow.com/
“Electric leg bag emptier 2”, http://www.wheelchairs.com/legbag2.htm
Melio Leg Bag website http://meliolegbag.com (no longer functional), earliest snapshot taken May 26, 2016, https://web.archive.org/web/20160523070322/http://meliolegbag.com/the-system/

People with moderate to severe disabilities driving wheelchairs commonly use internal and external catheters connected to a urinary bag and frequently rely on other people, who may not always be available, to empty their urinary bag. Not being able to empty the urinary bag before it gets full may lead to urinary tract infections due to the bag overflow and further cause the bursting of the urinary bag requiring a clean up and causing embarrassment to the disabled person. Due to the fear of urinary bag overflow, disabled people frequently decide to reduce liquid intake. Reduced liquid consumption can lead to a number of problems including dehydration, urinary tract infections, and crystal formation in urine for people using internal catheters. In addition, a disabled person using an external catheter suspecting that their urinary bag may be getting full may try to refrain from urinating by holding the urine in the bladder which too may lead to urinary tract infections. As such the ability to timely empty the urinary bag by a disabled person is not only a matter of convenience but also a health requirement.

A number of solutions have been suggested in the past to make it possible for a disabled person to empty their own bag. These solutions commonly include some form of a button or lever controlled valve installed at the output of the urinary bag. Disabled people are thus able to open the valve and empty the contents of their bag such as onto the ground or the floor drain in the bathroom. However an option of emptying the contents of a urinary bag onto the ground may not appeal to some disabled people and may not be an option for disabled people who are employed.

Some of the solutions suggested in the past also deployed the use of a pump to pump out the urine from the urinary bag. For example, in GB2496605A (the Melio bag) the pump is integrated with the leg bag and allows a person to pump out the contents of the urine bag into the toilet or any other receptacle. However, this approach is only useful for people with a substantial muscle control. Additionally the Melio bag implementation is expensive to use since the pump is permanently attached to the leg bag and is meant to be disposed of every time the urinary bag is replaced (which is typically several weeks to a month).

Another approach described in US6012181 uses a rigid extension tube on the side of the wheelchair which a disabled person could manually extend out and position over the toilet or urinal to subsequently actuate the pump and empty the contents of the urinary bag. Such an approach is only suitable for people who have enough muscle control to extend the rigid extension tube over the toilet. An approach similar to the one described in US6012181 was later implemented in “The PUMP” system.

Most systems described in the past provide solutions to be able to empty the urinary leg bags but do not offer ways to increase the amount of urine which can be stored before emptying is required. To address this limitation the use of an additional urine storage container mounted on a wheelchair was suggested in US6526603. That solution allows storing a larger amount of urine in a container attached to the wheelchair and suggests the use of a float or a sensor to monitor the amount of urine. However the solution in US6526603 uses of a pump to transfer urine from a leg bag to the larger storage container mounted on a wheelchair and suggests a once-a-day removal of urine from that container when the container clean-up is performed.

Several additional convenience features were implemented in the past including the usage of an output check valve in US6012181, the use of quick disconnects to connect the urinary bag to the emptying system in US6526603 and the “GoPee”, and powering the system from the wheelchair battery in “Kan-Go-Wee”.

The solutions suggested so far for disabled people to be able to independently empty the contents of their urinary bags into toilets or urinals are not suitable for disabled people with severe muscle control issues who have to use power wheelchairs. As such there is a need in an improved design of a urine evacuation device to be used on power wheelchairs targeting people with moderate to severe muscle control issues.

### BRIEF SUMMARY

Embodiments of the present invention relate to a device for unassisted emptying of urinary bags into sanitary bowls and urinals by disabled persons driving power wheelchairs equipped with tilt functionality. The urinary bag drainage device takes advantage of the driving and tilt capabilities of a power wheelchair to position the urinary bag drainage device outlet port over the toilet or urinal for emptying.

The drainage device is designed so that the outlet port is located near the front of the footplate. The outlet port contains a check valve. The urinary drainage device contains a pump to evacuate urine and a normally closed electrically actuated output valve connected to a rechargeable control module.

The device further includes two inputs to connect one urinary leg bag and one large capacity urinary bag. The urinary bags emptying device is equipped with sensors to monitor if the outlets of the two urinary bags are connected to their corresponding urinary bag drainage device inputs. The urinary leg bag is connected to a catheter and is placed on the leg of the disabled person. A large capacity urinary bag is mounted on the power wheelchair behind the footplates under the front part of the seat in the spacing between the footplate support mount and the front wheelchair main body to which the wheels are attached. An input filter is installed at the output of the urinary leg bag to reduce the amount of contaminants entering the urinary bags emptying device. Both outputs of the urinary bags are interconnected to let the urine flow from the leg bag to the large capacity bag due to gravity. The large capacity bag is hung lower than the leg bag to let the urine drain from the leg bag into the large capacity bag when both bags are connected to the urinary bag drainage device. Additionally the large capacity bag mounted on a wheelchair is hung as low as possible while providing at least the same amount of road clearance as other wheelchair components. The urinary bag drainage device can be used with both bags attached or only one of the two bags attached. The outputs of both urinary bags are connected to the input of the pump. The device further includes a weight sensor attached to the wheelchair and connected to the control module. The weight sensor includes hardware to hang the second large capacity urinary bag onto. The weight sensor is used to provide information about the amount of urine present in the second large capacity bag.

The control module includes a display to indicate the amount of urine in the large capacity bag measured by the weight sensor. The control module further includes an input for optional additional weight sensors which can be used to measure the amount of liquid left in additional one or more separate containers which may be attached to the wheelchair to contain water or other liquids used for drinking purposes. The control module is equipped with a built in accelerometer for the power wheelchair seat tilt angle measurement to aid in choosing the correct angle for outlet port aiming. The control module is equipped with visual and audio indicators to provide information form the sensors and the accelerometer as well as the battery charge status. The control module additionally includes a communication port to read values form the attached sensors. The control module further includes a port which allows actuating the pump and electric valve module externally. The control module is used to actuate the pump and the electrically actuated valve to empty the urinary bags. The actuation of the pump and the electrically actuated valve does not involve the use of the microcontroller circuit.

### DRAWINGS - FIGURES

FIG. 1 - Urinary bags drainage system

FIG. 2 - Urine evacuation module

FIG. 3 - Extension tube attachment with a filter

FIG. 4 - Attachment of the urine evacuation module onto a wheelchair

FIG. 5 - Placement of urinary bags and urinary bags emptying configuration

FIG. 6 - Variations in the installation of the pump and electric valve module on wheelchairs with central bar and side footplate supports

FIG. 7A - Control module front

FIG. 7B - Control module back

FIG. 8 - Mounting of control module under the armrest

FIG. 9 - Schematic diagram of the selected system electrical and hardware components and their interaction

FIG. 10 - Configuration of the tilt angle LED colors and wheelchair tilt angles

### DETAILED DESCRIPTION - FIRST EMBODIMENT

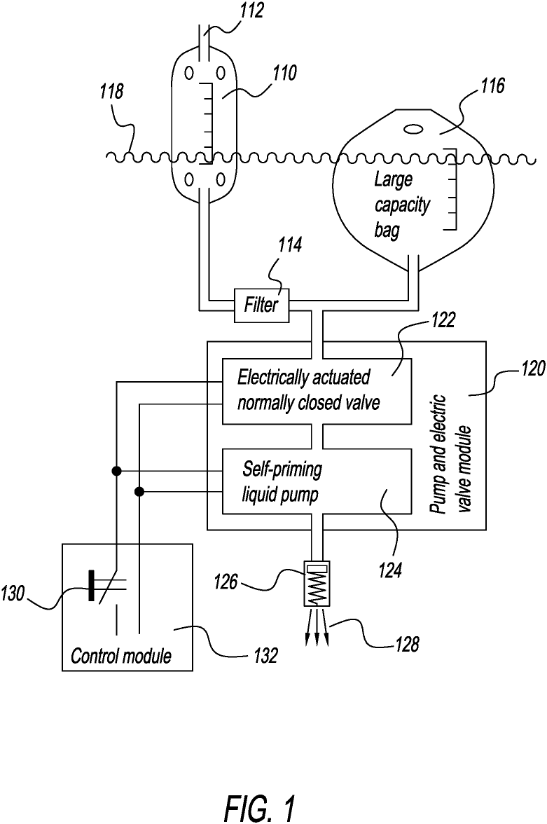

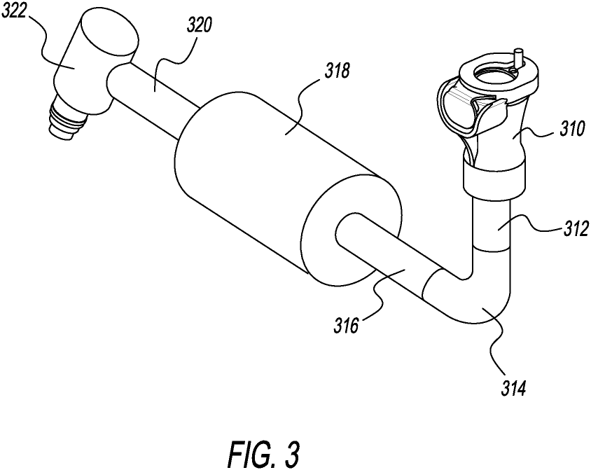

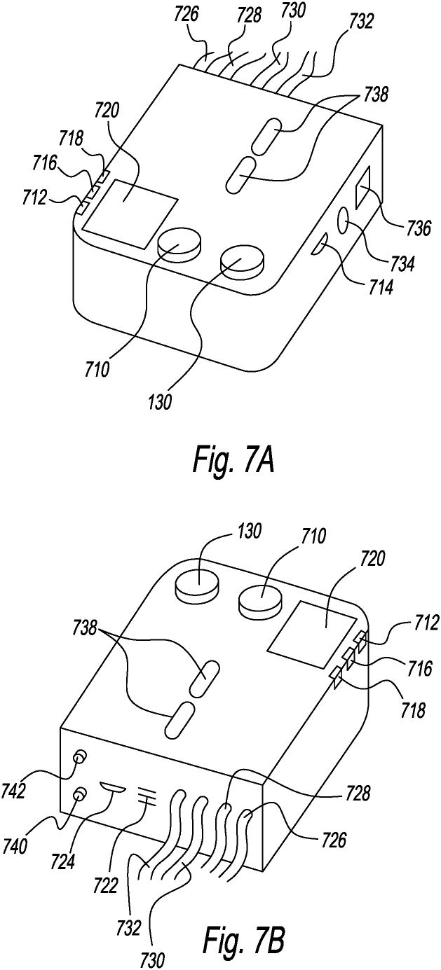

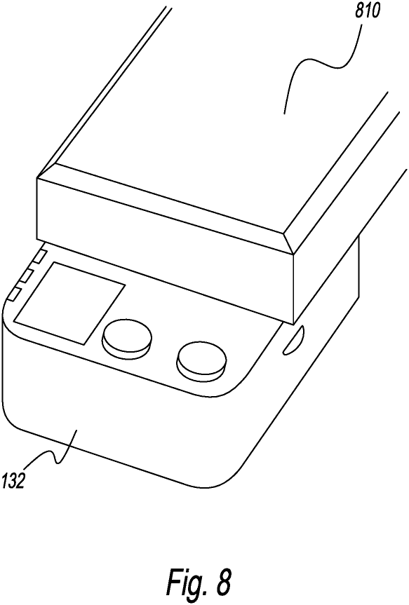

The urinary bags emptying system for power wheelchairs contains a urinary leg bag 110 as shown in FIG. 1. Leg bag 110 is to be attached to a disabled person’s leg. The urine flows into leg bag 110 through an inlet 112. The output of urinary leg bag 110 is connected to an optional input filter 114 to reduce the amount of contaminant sediment present in urine entering the rest of the urinary bag drainage device. The system further contains a large capacity urinary bag 116 which is mounted onto the power wheelchair. The smaller capacity leg bag 110 to be placed on a person’s leg is positioned higher than the large capacity urinary bag 116 attached to the wheelchair. The outputs of bags 110 and 116 are connected together allowing the urine from leg bag 110 to flow into the large capacity bag 116 under gravity so that the urine would be stored in the large capacity bag unless the amount of urine exceeds the maximum urine level 118 in the large capacity bag determined by the elevation of the leg bag 110 output relative to the elevation of the large capacity bag 116. The outputs of bags 110 and 116 are further connected to the input of the pump and electric valve module 120 which contains a normally closed electrically actuated valve 122, and a self-priming pump (such as the diaphragm pump) 124. The output of the pump and electric valve module 120 is connected to an output nozzle with a built-in check valve 126 which opens under pressure when valve 122 is open and pump 124 is enabled resulting in an output stream of urine 128 evacuating bags 110 and 116. Valve 122 and pump 124 are actuated simultaneously with an actuation button 130 located in the control module 132 also shown further in FIGS. 7 and 8. Filter 114 may be made to be a part of the pump and electric valve module 120 or as an external attachment as described further and shown in FIG. 3.

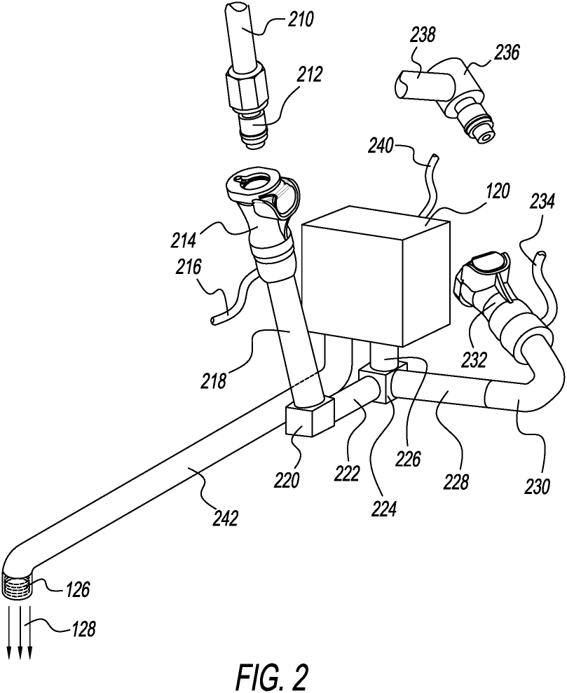

The construction of the urine evacuation module is depicted in FIG. 2. Output tubing 210 leading from the urinary leg bag is connected to an inline valved male quick disconnect coupling 212 which may be inserted into a female inline valved quick disconnect coupling 214 connected to tubing 218 leading to inlet 226 of the pump and electric valve module 120 through elbow right angle connectors 220 and 224 and tubing 222. Signal wire 216 is connected to a sensor attached to or built into quick disconnect 214 to determine if the male quick disconnect coupling 212 is inserted into the female quick disconnect 214. Wiring 216 is connected to a control module described further in FIG. 7. Right angle elbow connector 224 has 3 interconnected couplings attached to tubing 22, and 228. Tubing 228 is attached to the second female quick disconnect 232 though a curved coupling 230. The female quick disconnect 232 may be attached to the second high capacity urinary bag though a male quick disconnect 236 and tubing 238. Signal wire 234 is connected to a sensor attached to or built into quick disconnect 232 to determine if the male quick disconnect coupling 236 is inserted into the female quick disconnect 232. Wiring 234 is connected to a control module described further in FIG. 7. The urine evacuation module may be used with either one or both of the urinary bags attached. When the power is supplied to the pump and electric valve module 120 though cable 240 the urine is evacuated from any bags attached to either one or both of female quick disconnects 214 and 232. The output of the pump and electric valve module 120 is connected to outlet tubing 242 which is curved at the output to point down (close to or at the right angle) and is equipped with an output nozzle with a built-in check valve 126 attached at the output. The pump and electric valve module 120 is connected to the control module described further in FIG. 7. Wiring 216, 234 and 240 are connected to the control module shown in FIG. 7 as a bundle or separately. For convenience wiring 216 and 234 may be bundled to be transmitted over RJ-45 (8P8C) or other cable. Wiring 240 is used to supply power to the pump and the electric valve module to enable them when emptying is required. Wiring 216 and 234 is used to retrieve information from the quick disconnect connection sensors built-in or attached to the quick disconnects 214 and 232 respectively.

FIG. 3 depicts the extension tubing module containing the input filter element. Female quick disconnect 310 is connected to filter 318 with tubing 312, elbow connector 314, and tubing 316. Filter 318 is further connected to male quick disconnect 322 with tubing 320. The extension tubing filter module is meant to be installed between the input of the urination system and the leg bag output by connecting female quick disconnect 310 to the leg bag output quick disconnect 212 shown in FIG. 2, and by connecting the male quick disconnect 322 to female quick disconnect 214 shown in FIG. 2. The input filter may also be made a part of the pump and electric valve module 120 in which case the tubing 222 and the right angle elbow connector 224 would have to be re-arranged to make sure that the urine flowing though tubing 222 would pass though the filter prior to entering the right angle elbow connector 224 to prevent unfiltered urine from entering the large capacity bag 116 (FIGS. 1 and 2) and the rest of the urine evacuation module.

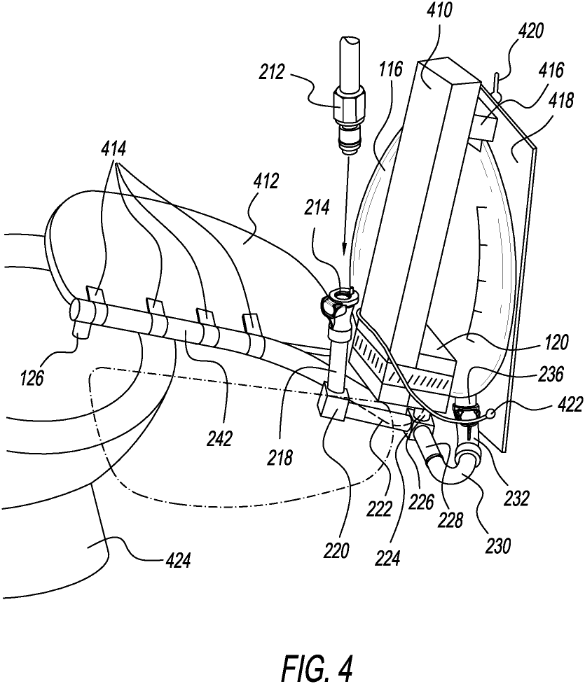

FIG. 4 depicts the attachment of the urine evacuation module to the power wheelchair footplate components. The pump and electric valve module 120 is secured to the footplate central support bar 410. The output tubing from the pump and electric valve module is attached to one of the footplates 412 with clamps 414 or other types of fasteners so that the output 126 is located at the front edge of the footplate. The front female quick disconnect 214 is connected to the male quick disconnect 212 attached to the leg bag on a person’s leg. The rear quick disconnect 232 is connected to the large capacity urinary bag 116 which is hung on a weight sensor 416 attached to the back of the footplate central support bar 410. The weight measurement provided by sensor 416 is used to monitor the amount of fluid in the urinary bag and is transmitted to the control module (shown in FIG. 7) to be displayed to the user. A support plate 418 hanging on a swivel mount 420 is attached behind the large capacity urinary bag 116 and is secured with a thread 422 to the central bar footplate support 410 to maintain the support plate and the large capacity bag in place when the wheelchair is tilted. The wheelchair driving and tilt functions are used to position the output nozzle with a built-in check valve 126 over the toilet bowl 424 or urinal.

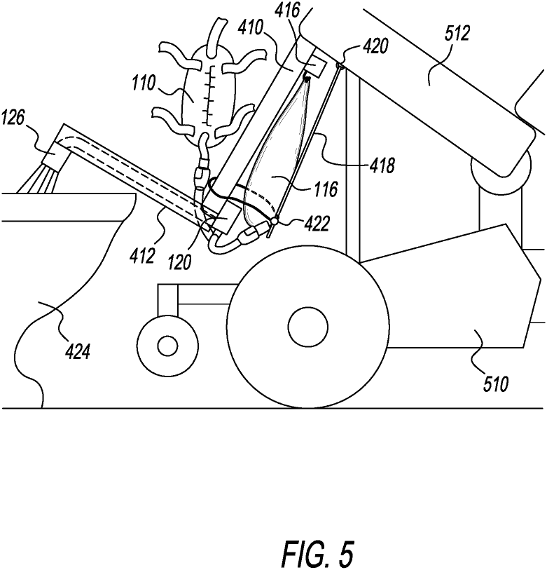

FIG. 5 shows the placement of urinary bags and the configuration to empty them. The wheelchair 510 is tilted and driven close to the toilet bowl 424 so that the output nozzle with a built-in check valve 126 located at the front edge of a foot plate 412 is over the toilet bowl for emptying. The urine is pumped from leg bag 110 and the large capacity bag 116 by the pump and electric valve module 120. The large capacity bag 116 is hung onto the weight sensor 416 attached to the footplate central beam support bar 410 under the front side of the seat 512. The weight measurement provided by sensor 416 when the wheelchair is in horizontal position (not tilted) is used to monitor the amount of fluid in the urinary bag and is transmitted to the control module (shown in FIG. 7) to be displayed to the user. To prevent the large capacity bag from falling when the wheelchair is tilted a support plate 418 is attached behind the large capacity bag by means of a swivel mount 420 and thread 422 which goes around the central beam footplate support bar 410 or attached to the central beam support bar by other means.

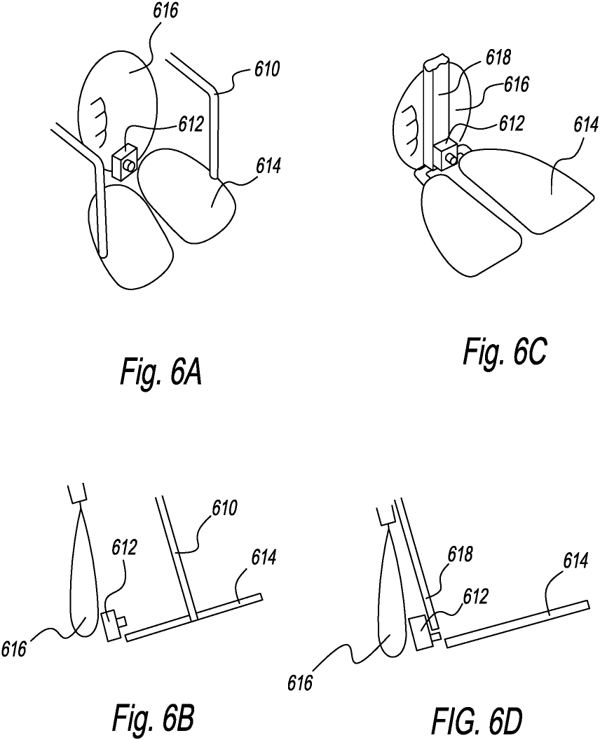

FIG. 6 shows differences in the placement of the pump and electric valve module as well as of the large capacity bag onto wheelchairs equipped with the side foot plate supports 610 (FIGS. 6A and 6B) versus the central beam foot plate support 618 (FIGS. 6C and 6D). As shown in FIG. 6, regardless of the footplate support configuration the pump and electric valve module 612 is mounted near the inner back corner of the footplate 614 or immediately behind the inner back side of the footplate. For wheelchair models with side footplate supports 610 (FIGS. 6A and 6B) the pump and valve module 612 may be attached to the footplate 614 itself instead of to the central beam support 618 (FIGS. 6C and 6D). Such pump placement corresponds to the lowest elevation in the emptying system as shown in the side view FIGS. 6B and 6D when the wheel chair is tilted for emptying thus driving the liquid located in the urination bag into the pump by gravity in addition to the suction of the pump. Additionally, depending on the central beam support configuration the pump and electric valve module may be attached either at the front of the central beam support (FIG. 6C), or at the back of the central beam support (FIG. 6D). The large capacity bag 616 is placed behind the central beam footplate support (FIGS. 6C and 6D) for the systems with the central beam support configuration or hung under the front of the wheelchair sit behind the footplates as shown in FIGS. 6A and 6B for wheelchairs equipped with side footplate supports.

FIG. 7 shows the control module. A momentary contact push button 710 is used to turn the unit on. LED 712 is turned on when the power of the unit is turned on with button 710. The unit is equipped with an automatic timed idle shut off feature after a predefined time if not in use. Manual unit turn off is also possible by pressing and holding button 710 until the power LED 712 is turned off. A momentary contact push button 130 is used to turn on the pump and open the output valve. Button 130 must be pressed and held pressed in for as long as the pump is to be on and the output valve is to be open during emptying. Once button 130 is released the pump will stop its operation and the output valve will close. The control unit is supplied with a charging circuit and a rechargeable battery which is charged through the micro-USB or other type of connector 714. The control unit further contains 3 light emitting diodes (LEDs): the power-on indicator LED 712, the 3 color “red - yellow - green” LED 716 used to indicate the tilt angle of the wheelchair measured with an accelerometer built into the control module, and LED 718 which is turned on when the rechargeable battery is charging. An E-ink or other type of display 720 is used to display the system state information including the amount of urine in a large capacity urinary bag, the battery charge level as well as if the urinary bag valved male quick disconnects 212 and 236 are connected to the valved female quick disconnects 214 and 232 (FIG. 2). The control box is supplied with a piezoelectric buzzer or speaker 722 to provide audible alarm for selected events such as when the fluid level in the bag approaches the set maximum. The speaker may also be optionally used to indicate the tilt angle as an alternative to or in addition to the visual 3-color LED indicator 716. The location of the buzzer may be at the back of the unit or any other location within the unit. The control module further contains the second charging port 724 at the back of the unit. The control unit wiring 726 shown in FIGS. 7A and 7B is connected to the pump and electric valve unit wiring 240 depicted in FIG. 2 and is used to turn on the pump and open the electronic valve. Wiring 728 is a bundle connected to wiring 216 and 234 (FIG. 2) connected to quick disconnect connected state sensors built into quick disconnects 214 and 232. Wiring 730 is connected to the weight sensor 416 (FIG. 4 and FIG. 5). The control module is able to display the information from an additional one or several weight sensors and is equipped with an additional extra input 732 to retrieve information from additional weight sensors. The control module further contains connector 734 to enable the possibility to actuate the pump and electric valve module externally, and a communication port 736 connector to be able to communicate with the microcontroller and read the information from the sensors connected to it. The control module enclosure contains the mounting holes 738 at the top of the unit. The minimum and maximum set buttons 740 and 742 are used to record the reference minimum and maximum urine amounts in the urinary bag into the control unit internal memory and/or to set the minimum and maximum angles of acceptable tilt range resulting in better placement of the drainage device output over the toilet receptacle.

FIG. 8 shows the attachment fo the control module enclosure 132 under the front of the armrest 810. Due to design differences some wheelchair models do not allow installation of a square control box, in which case an L-shaped or other shape control unit enclosure may be necessary. The control unit 132 shown in FIG. 8 is for installation under the left armrest. The location of the controls on the control unit may be mirror inverted for convenience relative to central plane if installation under the right armrest is required.

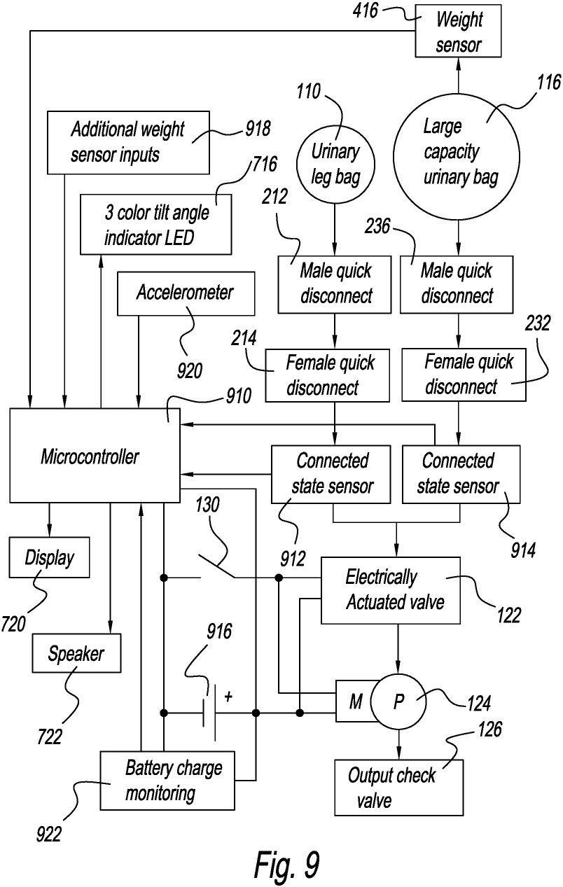

FIG. 9 presents a diagram illustrating the selected electronics and hardware components of the urinary bags emptying system and their interaction. The large capacity bag 116 is attached to a weight sensor 416. The information form the weight sensor 416 about he weight of the large capacity bag 116 is communicated to a microcontroller 910. The microcontroller 910 is attached to a display 720 and a speaker 722. The outputs of the leg bag 110 and the large capacity urinary bag 116 have male quick disconnects 212 and 236 attached to their drain tubes. The male quick disconnects 212 and 236 are to be attached to the female quick disconnects 214 and 232 respectively. The female quick disconnects 214 and 232 are equipped with connected state sensors 912 and 914. The connected state sensors 912 and 914 are connected to the microcontroller 910. The output of female quick disconnects 214 and 232 are connected to each other and to the input of the electrically actuated value 122. The output of the electrically actuated valve is connected to the input of the pump 124. The output of the pump is connected to the output check valve 126. The electrically actuated valve 122 and the pump 124 are enabled by supplying power from battery 916 by closing switch 130. The supply of power to the electrically actuated valve and the pump is carried out by a circuit independent of the microcontroller circuit meaning they operate using the same battery however actuating the pump 122 and the valve 124 does not require the microcontroller 910 to be turned on. Additional weight sensors 918 may be connected to microcontroller 910. An accelerometer 920 is connected to the microconroller 910 to provide the information about the wheelchair tilt angle which is displayed to the user though the 3 color tilt angle indicator LED 716. The tilt angle indicator light emitting diode 716 is connected to a microcontroller 910 and is used to help the disabled person achieve correct tilt angle to avoid possible spills on the bathroom floor due to imprecise aiming of the drainage device output over the toilet bowl. A battery charge monitoring circuit 922 is is used to monitor and relay the remaining charge information to the microcontroller. The microcontroller circuit and other electronic components are powered by the same battery 916.

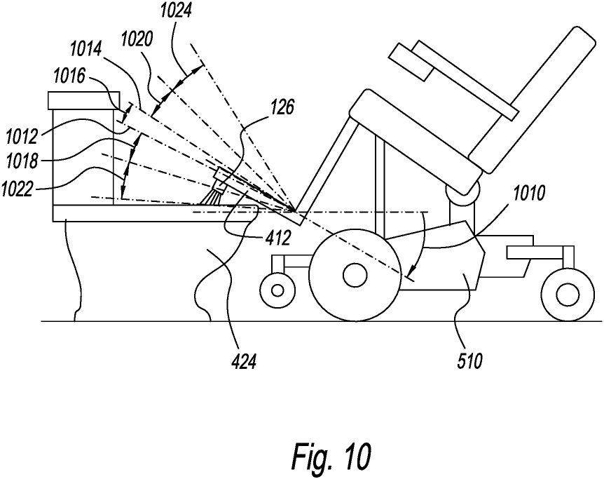

FIG. 10 illustrates the configuration of the tilt angle LED colors in reference to the wheelchair tilt angles. The tilt angle indicator LED 716 (FIG. 7) has 3 colors (red, yellow, and green) to display the proximity of the current tilt angle to the range of optimum tilt angles between the minimum and maximum programmed values as shown in FIG. 10. The tilt angle within the set minimum to maximum range optimum for correct positioning of the output nozzle over the toilet bowl for emptying will be indicated by a green color of the tilt angle LED. Higher and lower tilt angles adjacent to the green regions would be indicated by a yellow light emitting diode color and then by the red color if the tilt angle is increased or decreased further from the green and yellow region tilt angles as shown in FIG. 10. The angle spans corresponding to the yellow and red LED light angles may be set larger than the manually programmed “minimum - maximum” angle span corresponding to the green color of the LED light. Keeping the angle spans the same for different tilt angle indicator LED colors is also possible but makes it more difficult to monitor the tilt angle changes due to fast switching between different colors as the tilt angle is changed.

### Operation

The urine emptying procedure operation details are as follows. The disabled person would drive to the bathroom when the urinary bag needs emptying based on a physical perception and/or the information provided by the urinary bag drainage system via its display 720, LEDs 716 and/or the audible alarms from speaker 722 based on the data from weight sensor 416 measuring the amount of urine in the large urinary bag. If the urinary bag drainage system (FIG. 2) is not connected to one (110, 116) or all urinary bags as detected by sensors built into the quick disconnects 214 and 232, the system would notify the user who would need to contact a caregiver to have the system connected. To empty the bag a disabled person would drive the power wheel chair into a bathroom. In the bathroom the disabled person would first turn on the control module unit by pressing button 710 (FIG. 7) which would power on the unit and enable the tilt angle 3-color LED 716 (FIG. 7) in the control module. The unit would shut off automatically if not in use after a predefined time has elapsed or may be turned off manually by pressing and holding the power button 710 until the power LED 712 (FIG. 7) turns off. The unit would not auto shut off when the tilt angle 3-color LED 716 is lit regardless of the color (green, yellow, or red). While the control unit is powered, the user would first enable the tilt functionality of the wheelchair and tilt the wheelchair seat along with the attached footplates so that the urinary bag drainage device outlet 126 (FIGS. 2 and 4) is lifted high enough to be placed over the toilet bowl or the urinal. In tilting the wheelchair to the desired angle the user would use his visual perception and/or the tilt angle light emitting diode indicator 716 (FIG. 7) to achieve an angle which is high enough to place output 126 (FIGS. 2 and 4) over the toilet but not too high to drive too far and miss the toilet bowl. Once the desired tilt angle has been achieved, the user would drive the tilted wheel chair to position the outlet 126 closer to and over the toilet bowl or the urinal by using his visual perception. The user may have to alternate the use of the tilt and driving functionality of the power wheelchair repeatedly until proper position over the toilet bowl or urinal is achieved. Such alignment may also be performed without turning on the control module 132 (FIG. 8) and without using the tilt angle 3-color LED indicator 716 (FIG. 7). Once the alignment is complete, the user would enable the power of the control unit by pressing power button 710 (FIG. 7) if not on already, and then open the electrically actuated valve and enable the pump at the same time by pressing the urine release button 130 (FIGS. 1 and 7). The user would hold button 130 (FIG. 7) at all times the urine is to be pumped out until the contents of the urinary bag have been completely evacuated or evacuated to the desired level. Once the emptying is complete the disabled person would release button 130 (FIG. 7), drive back away from the sanitary bowl or urinal, then return the tilt angle of the wheelchair down to the normal position and drive out of the bathroom. Upon completion of the emptying procedure and returning the wheelchair to its driving position the control unit would automatically shut off after a predefined idle auto-shutoff period has elapsed. Turning the unit off manually is also possible by pressing and holding button 710 (FIG. 7) until the power LED 712 (FIG. 7) is turned off.

The control module contains a microcontroller as depicted in FIG. 9. The battery charge state is constantly monitored by the microcontroller by means of a charge level monitoring circuit 922 connected to the rechargeable battery. The piezoelectric speaker 722 is used to notify the user if the amount of urine in the large urinary bag is or nearing its maximum or reaches other pre-programmed levels as well as of other events such as the low battery charge level. The microcontroller circuit is constantly on. The monitoring of the urine weight in the large capacity bag using the weight sensor 416 as well as the state of the connected state sensors built into quick disconnects 214 and 232. is carried out by the microcontroller at all times even when the control module 132 is not switched on using button 710 (and LED 712 is not lit). However the tilt angle 3-color LED 716 is switched on only when the charging and power unit 132 is manually switched on regardless of if the wheelchair is tilted or not. After the control module 132 is manually switched on, for as long as the tilt angle 3-color LED 716 is lit (when the chair is tilted) the charging and power unit will stay on even if the predefined auto shut off time has passed, unless the control unit is manually switched off by pressing and holding button 710 (FIG. 7). The piezoelectric buzzer or speaker 722 would sound an alarm if the liquid level in the bag is approaching or has reached the preset maximum or selected intermediate levels. The control unit is supplied with an additional input 732 (FIG. 7) to retrieve information from additional weight sensors.

Buttons 740 and 742 on the control unit (FIG. 7) have multiple functions depending on if the quick disconnects 214 and 232 (FIG. 2) are connected to their respective quick disconnects 212 and 236. If the quick disconnects 214 and 232 are connected to quick disconnects 212 and 236, buttons 740 and 742 are used to record the reference minimum and maximum urine amounts in the urinary bag into the control unit internal memory respectively. To record the minimum reference urine amount level button 740 is pressed when the urinary bag has the lowest amount of urine the user considered by the user to be empty or another minimum value of choice. To record the maximum reference fluid level button 742 is pressed when the urinary bag has the largest amount of fluid the user considers to be the acceptable maximum. When either of buttons 740 or 742 is pressed the corresponding minimum and maximum values measured by the weight sensor 416 (FIG. 4) are recorded into the microcontroller non-volatile memory which is preserved when the power is turned off.

To better aim the drainage device output over the sanitary bowl the wheelchair tilt angle should be high enough to be able to drive the output 126 (FIGS. 2 and 4) over the toilet bowl but not too high to avoid possible driving too far over the toilet receptacle. To facilitate the correct angle selection an accelerometer is used. To use this functionality the user would have to perform a setup the first time the system is used to program the minimum and maximum angles of acceptable tilt range resulting in better placement of the drainage device output over the toilet receptacle. These minimum and maximum tilt angles are programmed by pressing the minimum 740 and maximum 742 (FIG. 7) tilt angle programming buttons as follows. When quick disconnects 236 and 232 (FIG. 2) are disconnected, the minimum 740 and maximum 742 buttons are used to set the minimum and maximum tilt angles respectively which are used to indicate the optimum angles using LED 716 (FIG. 7) for positioning the wheelchair over the toilet bowl for emptying. Once button 740 (FIG. 7) is pressed when the unit is switched on and the quick disconnects 236 and 232 (FIG. 2) are disconnected, the currently measured tilt angle is programmed into the microcontroller 910 (FIG. 9) non-volatile memory corresponding to the minimum tilt angle. Button 742 is used in the same manner to record the maximum tilt angle value into the microcontroller non-volatile memory. Buttons 740 and 742 (FIG. 7) operate by recording the current wheelchair tilt angle measured by the accelerometer 924 (FIG. 9) in the microcontroller non-volatile memory for later reuse. Once the minimum and maximum tilt angles are set, the tilt angle proximity to the optimum tilt angle range would be indicated using the 3-color LED 716 (FIG. 7). The programmed minimum and maximum angles will correspond to the boundary angles of the region optimum for aiming purposes corresponding to the green color of the LED 716 as shown in FIG. 10.

### Advantages

From the description above, a number of advantages of some embodiments of my urinary bags draining device become evident:
the outlet is mounted near the front of the footplate and relies on motion and tilt capabilities of a power wheelchair to position the outlet over the sanitary bowl or urinal.
the current device allows frontal facing of the sanitary bowl or the urinal by a disabled person which is needed when using public restrooms and urinals with stalls.
the pump is mounted near the back or behind the back of the footplate which corresponds to the lowest elevation in the urinary bag and emptying system when the wheel chair is tilted for emptying.
uses quick disconnect components to quickly connect/disconnect the emptying system to/from the urination bags.
contains an input filter to remove sediment from urine to reduce and slow down contamination of the urinary bags draining device
utilizes a circuit with an accelerometer to facilitate in tilting the power wheelchair footplates to the optimum angle for outlet positioning over the emptying receptacle (toilet bowl or urinal).
visual and/or audio indicators may be used to notify the user of the tilt angle measured with an accelerometer.
a weight sensor is used to hang the large capacity bag onto and measure and display the amount of urine in the large capacity urinary bag.
uses two urinary bags one leg bag and one large capacity bag.
two urinary bags are positioned at different heights to let the urine drain into the large capacity bag from the leg bag to keep the leg bag empty. Keeping the leg bag empty is more comfortable for a disabled person and more convenient for caregivers during transfers in and out of the wheelchair.
two sensors for both bags are built into the device to notify the user if the urinary bags are connected to the urinary bag drainage device with quick disconnects.
automatic control unit power shutoff (with the exception of the microcontroller monitoring circuits) after a predefined time interval if not in use.

### Conclusion, Ramification, and Scope

Thus the reader will see that at least one embodiment of the urinary bags draining device for power wheelchairs is more convenient for both disabled people and caregivers. It provides an independent way for people driving power wheelchairs to empty the contents of their urinary bags into toilet receptacles and urinals by using the tilt and driving functions of a power wheelchair. Additionally at least one embodiment of the device allows keeping the urinary leg bag empty which is more comfortable. Sensors and electronics are provided for monitoring purposes ensuring the urine bags are connected, and making urine bag overflow less likely.

While my above description contains many specifities, these should not be construed as limitations on the scope, but rather as an exemplification of the [or several] embodiment(s) thereof. Many other variations are possible, for example
the electrically actuated valve may use different types of electronic actuation mechanism i.e. electromagnetic, electric motor, or a stepper motor based,
if the pump blocks the liquid flow through the urine draining device when the device is not powered then the electromagnetic valve does not have to be present,
the electromagnetic valve may be placed before or after the pump,
the control module may have a wireless connection to the emptying system (containing the pump, and the electronically actuated output valve). In this case the pump and valve module as well as the weight sensor would be supplied with their own additional power source,
the output may be mounted under the footplate if the clearance between the footplate and the ground allows this,
the tilt angle indication LEDs may have colors different from red, green and yellow and may be programmed to indicate the tilt angles differently,
the urinary bag may be connected to a female quick disconnect while the pump and electromagnetic valve units are connected to a male valved quick disconnect coupling,
the control module may be replaced with alternative control methods for people with limited abilities (i.e. to react to head movements etc.),
multiple programmable tilt angle values for different tilt toilet bowl / urinal etc. height levels may be enabled in the control unit,
if the footplates are attached with side mounting supports, the pump may be attached to the back of the footplate itself or else to one of the side footplate supports,
the pump may be rotated 90 degrees so that the inlet / outlet tubing is routed from the left or right side of the pump instead of under the pump if not enough clearance is available between the wheelchair footplate hardware and the ground,
the system may be used with an internal catheter or an external catheter,
the display used in the control module may be an e-ink or any other type of a display,
the tilt angle may be additionally displayed as a numerical value on the control module display,
the charging connector may be a micro-USB or any other type of connector,
wiring connecting the control module (FIGS. Fg. 7) with the urine evacuation module (FIG. 2) and the weight sensor(s) may be bundled differently as a single cable or any other combination of wire bundles,
the piezoelectric buzzer or the speaker may be located anywhere within the system,
the buttons, LED indicators, the display or any other control module components may be arranged differently in the control module,
the shape of the control module may be different,
the large capacity night bag may be placed anywhere on the chair,
the large capacity bag may be replaced with a canister,
the quick disconnects may be supplied with plugs to block them when they are disconnected to avoid spills if the seal gets damaged,
extra tubing may be introduced between coupling 230 and the quick disconnect 232 in FIG. 2,
the input and output to/from the pump and electric valve module may be routed differently,
the combination of tubing and couplings leading to and from the pump and electric valve module (FIG. 2) may be different,
the large capacity bag may be attached to the weight sensor with auxiliary hardware,
the LEDs may be used in addition to the display to notify the user of the urine amount reaching predefined levels in the urinary bag including when nearing the maximum capacity,
tubing 242 (FIG. 2) may be attached to the footplate in alternative ways including by using velcro fasteners,
the extension tubing module (shown in FIG. 3) may have no filter attached to it,
either left or right foot plate may be used to attach the urinary system output tubing to,
the weight sensor may be used in combination with the accelerometer tilt angle data to estimate the weight of urine in the large bag even when the chair is tilted,
the control module may be installed under the left or right armrest,
controls on the control module may be mirror inverted for left/right armrest control module installation,
the pump may be a diaphragm pump of any other type of self-priming pump that is capable of pumping liquid and air,
a power output port (i.e. USB) may be added to the control module,
the input filter may be made a part of the pump and electric valve module 120 instead of as an attachment between the leg bag and the input quick disconnect component as shown in FIG. 3,
the urine drainage device may contain more than one large capacity bags,
the pump and the electric valve may be mounted at different locations by using longer input and output tubing lines,
the functionality of the min / max buttons may be different in the way the programming is done, i.e. relative to the status of the quick disconnect sensors,
the control module may be built into the power wheelchair electronics.

Accordingly, the scope of the disclosure should be determined not by the embodiment(s) illustrated, but by the appended claims and their legal equivalents.

I claim:

1. A system for storage and emptying bodily waste from a leg bag carried by an occupant of a motorized wheelchair equipped with a motorized tilt, the leg bag including a drain tube, comprising a combination:
a large capacity urinary bag mounted on said motorized wheelchair substantially lower than said leg bag, the drain tube of said large capacity urinary bag being used as both an inlet and outlet and being connected to the drain tube of said leg bag;
a self-priming pump being mounted onto said motorized wheelchair, said self-priming pump having one discharge port and one inline port, said inline port being connected to the drain tube of said leg bag and the drain tube of said large capacity urinary bag;
a discharge line connected to the discharge port of said self-priming pump, the output of said discharge line being firmly mounted onto wheelchair footplate, footplate support or any hardware attached thereto;
a normally-closed check valve installed at or near the output of said discharge line;
means for actuating the said self-priming pump.

2. The system according to claim 1, wherein the connections of said leg bag drain tube and said large capacity urinary bag drain tube to the inlet port of said self-priming pump are equipped with quick disconnect means.

3. The system according claim 2, wherein said quick disconnect means are equipped with sensors having means of determining if said leg bag drain tube and said large capacity urinary bag tube are connected to the inlet port of said self-priming pump.

4. The system according to claim 1, wherein the quick disconnect means are equipped with an o-ring made of silicon rubber.

5. The system according to claim 1, wherein said discharge line is routed along the inner edge of said motorized wheelchair footplate, said output of said discharge line being mounted near the inner front corner of either wheelchair footplate.

6. The system according to claim 1, further including an inline filter connected between the drain tube of said leg bag and the inlet port of said self-priming pump.

7. The system according to claim 1, further including a weight sensor mounted onto the motorized electric wheelchair having a means of attaching the large capacity night urinary bag to said weight sensor.

8. The system according to claim 1, wherein said large capacity urinary bag is mounted behind the wheelchair footplate supports under the wheelchair seat.

9. The system according to claim 1, wherein a collection container is used in place of the large capacity bag, said collection container having a tube connector attached near the bottom of said collection container, said tube connector being used as both an inlet and outlet.

10. The system according to claim 1, further including a normally closed valve being opened during the actuation of said self-priming pump, said normally closed valve being positioned before the inlet of said self-priming pump or after the discharge port of said self-priming pump.

11. The system according to claim 1, wherein said discharge line is equipped with a spout at the output.

12. An electrical control module for the bodily waste emptying system comprising a combination:
a means to actuate the pump;
a microcontroller;
an accelerometer means to establish the tilt angle of said wheelchair seat;
a means to relay the tilt angle of said wheelchair and the information from electrical sensors to the electric wheelchair user.

13. A system according claim 12, further including a means to connect to and obtain measurements from one or a plurality of weight sensors.

14. A system according claim 12, further including a means to determine if the leg bag drain tube is connected to the urine evacuation module.

15. A system according claim 12, further including a means to determine if the large capacity bag drain tube is connected to the urine evacuation module.

16. The system according to claim 12, wherein a 3-color combination of green, yellow and red colors is used to indicate the proximity of the motorized wheelchair seat tilt angle to the optimum tilt angle.

17. The system according to claim 12, wherein the system according claim 12 is made a part of the motorized wheelchair electronics.

18. The system according to claim 13, wherein the system according claim 13 is made a part of the motorized wheelchair electronics.

19. A method for aiming the output of a bodily waste discharge line over a sanitary bowl or urinal, said output of the discharge line being mounted on the wheelchair, comprising the use of the tilt and driving functions of the power wheelchair to elevate and position the output of the bodily waste discharge line over the toilet bowl or urinal.

20. The method according to claim 19, wherein the step of positioning of the output of the discharge line over the toilet bowl or urinal comprises the use of a means to measure the wheelchair seat motorized tilt angle.
